(19) United States
(12) Patent Application Publication
(10) Pub. No.: US 2013/0060166 A1
(43) Pub. Date: Mar. 7, 2013

(54) DEVICE AND METHOD FOR PROVIDING HAND REHABILITATION AND ASSESSMENT OF HAND FUNCTION

(71) Applicants: Nizan Friedman, Irvine, CA (US); Mark Bachman, Irvine, CA (US); David J. Reinkenmeyer, Irvine, CA (US)

(72) Inventors: Nizan Friedman, Irvine, CA (US); Mark Bachman, Irvine, CA (US); David J. Reinkenmeyer, Irvine, CA (US)

(73) Assignee: THE REGENTS OF THE UNIVERSITY OF CALIFORNIA, Oakland, CA (US)

(21) Appl. No.: 13/481,685

(22) Filed: May 25, 2012

Related U.S. Application Data
(60) Provisional application No. 61/530,335, filed on Sep. 1, 2011.

(51) Int. Cl. A61B 5/103

(52) U.S. Cl. 600/595

(57) ABSTRACT

The various embodiments provided herein are generally directed to a method, system, and/or device for providing rehabilitation and assessing function from a portion of the human body. In one embodiment, there is disclosed a method, system, and/or device for providing rehabilitation and assessing of hand function using an audio interface. The audio interface may be a music-based interface and device may include a monitor unit, such as a hand monitoring unit, for providing data of a movement to a computing device, such as a microcontroller. The computing device may output data to a music-based interface.

This application claims priority from U.S. Provisional Appln. No. 61/530,335 filed on Sep. 1, 2011. The entire contents of this application are incorporated herein by reference.

This invention was made with Government support under Grant No. HD062744 awarded by the National Institutes of Health. The Government has certain rights in this invention.

### BACKGROUND OF THE INVENTION

1. Field of the Invention

The present invention relates to a device, system, and/or method for providing physical therapy to various parts of the human body including providing for the rehabilitation and assessment of appendages using an audio interface.

2. Background.

Stroke remains the leading cause of chronic adult disability in Western countries with over four million survivors currently living in the United States. Following the onset of stroke, patients may receive several weeks of intensive rehabilitation in an attempt to increase cognitive and functional abilities. Through intensive and repetitive motion training, patients may be able to regain lost function through processes such as neural re-organization.

Unfortunately, the length of stay at inpatient rehabilitation facilities may be limited to two weeks and follow-up outpatient therapy is also often limited. Accordingly, patients must continue therapy independently at home without access to specialized equipment and supervised therapy. For example, an important task for patients with hand impairment is to repeatedly practice making the coordinated finger and thumb movements that humans use to manipulate objects, such as pincer grip, key-pinch grip, and finger-thumb opposition. Rehabilitation therapists and scientists believe such movements are important for patients to practice because they are the movements that patients must master to be able to use the hand to manipulate objects in daily life. Further, motor learning research has shown that motor learning does not transfer well to other tasks besides the ones practiced. In addition, motor learning and rehabilitation science has shown that patients must practice such movements thousands of times to reach the skill level that they have the potential to achieve.

Unfortunately, without the presence of a clinician, this type of task is neither engaging nor does it provide any quantitative measure of improvement. As a result, patients typically lose motivation to perform independent rehabilitation. Without ongoing practice in using the hand, individuals experience declines in motor function, including a decrease in hand motor ability that affects their ability to perform activities of daily living (“ADL”). What is needed and is not currently available is a device and method that allows patients with hand impairment to practice on individually, for example, thousands of times, the gripping movements required to manipulate objects in a motivating environment, which provides quantitative feedback.

Participating in music is a promising avenue for therapy due to its highly motivating and repetitive nature, two of the most important factors for regaining hand function. Furthermore, listening to music after a neurologic event may induce plastic changes in the cortical regions as well as increase attention span, neuropsychological scores, cognitive function and well-being.

### SUMMARY

The various embodiments provided herein are generally directed to a method, system, and/or device for providing rehabilitation and assessing function from a portion of the human body. In one embodiment, there is disclosed a method, system, and/or device for providing rehabilitation and assessing of hand function using an audio interface. The audio interface may be a music-based interface and device may include a monitor unit, such as a hand monitoring unit, for providing data of a movement to a computing device, such as a microcontroller. The computing device may output data to a music-based interface.

The at least one hand movement may include one of a pincer grip, key-pinch grip, and/or finger-thumb opposition. The music-based interface may be configured to communicate with one of a mobile device, a laptop, and a computer.

The music-based interface may configured to provide one of visual and audio feedback when at least one hand movement corresponding to a musical note is completed within a target window of time corresponds to a playing of the musical note by the music-based interface.

The hand monitoring may include a glove configured with a plurality sensors, and further may include electrical leads configured on one or more fingertips of the glove. In one embodiment, the music-based interface is configured as a MIDI interface, and may be configured to one of quantify, record, and store the one or more electrical signals from the hand monitoring unit.

The music-based interface may be further configured to provide an assessment of sensory motor ability through one of audio and video feedback. Further, the hand monitoring unit may be configured to translate data into a USB HID Keyboard command.

A method for providing rehabilitation is also disclosed and includes providing a hand monitoring unit configured with one or more electrical signals associated with a completion of at least one hand movement, and providing a music-based interface configured to receive the one or more electrical signals from the hand motoring unit representative of the at least one hand movement.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 is a schematic diagram of a music-based rehabilitation system in an embodiment.

FIG. 2 is a diagram showing an embodiment of the hand monitoring unit.

FIG. 3 is a diagram showing an example of function grips being trained and an example of the music-based interface.

FIGS. 4-6 show an example of a patient playing a computer game.

FIG. 7 illustrates open palm glove design in an embodiment.

### DETAILED DESCRIPTION

Each of the additional features and teachings disclosed below can be utilized separately or in conjunction with other features and teachings to provide a device, system, and/or method for providing rehabilitation and assessment of body function. Representative examples of the present invention, which examples utilize many of these additional features and teachings both separately and in combination, will now be described in further detail with reference to the attached drawings. This detailed description is merely intended to teach a person of skill in the art further details for practicing preferred aspects of the present teachings and is not intended to limit the scope of the invention. Therefore, combinations of features and steps disclosed in the following detail description may not be necessary to practice the invention in the broadest sense, and are instead taught merely to particularly describe representative examples of the present teachings

Moreover, the various features of the representative examples and the dependent claims may be combined in ways that are not specifically and explicitly enumerated in order to provide additional useful embodiments of the present teachings. In addition, it is expressly noted that all features disclosed in the description and/or the claims are intended to be disclosed separately and independently from each other for the purpose of original disclosure, as well as for the purpose of restricting the claimed subject matter independent of the compositions of the features in the embodiments and/or the claims. It is also expressly noted that all value ranges or indications of groups of entities disclose every possible intermediate value or intermediate entity for the purpose of original disclosure, as well as for the purpose of restricting the claimed subject matter.

Devices, methods, and/or systems are described which may provide therapy to a patient that may facilitate and motivate at home and clinic based practices. One embodiment includes a monitoring unit, such as a hand monitoring unit, an interface to an audio interface, such as a music-based interface, and an assessment system. In other embodiments, the hand monitoring unit may be replaced with other similar devices for other appendages of the body including the wrist, arm, core, and/or legs.

A form of hand therapy may use music as an interactive and motivating medium to guide hand exercise and to quantitatively assess hand movement recovery. A user may practice functional movements, which may include pincer grip, key-pinch grip, and/or finger-thumb opposition. These movements may be quantified using a hand monitoring device and used and/or configured as a controller to play musical notes within a music-based application, such as an interactive computer game.

The hand monitoring unit may include a glove configured with sensors. One embodiment may include a glove with one or more electrical leads configured on one or more fingertips of the glove. An electrical lead may also be configured on the proximal interphalangeal joint on the lateral aspect of the index finger. When the lead on the thumb touches one of the other leads, an electrical connection is closed and may be interpreted by a computing device, such as a computer or microcontroller, as an event. The leads may be configured in any way that is capable of forming an electrical connection and being interpreted as an event. In other embodiments, flex or pressure sensors may be used. In another embodiment, a glove with an open palm design allowing a person with hand impairment to more easily don and doff the device.

Data from the hand monitoring unit may be provided to a microcontroller or similar computing device. The microcontroller may analyze the received data and provide an output to a music-based interface. In one embodiment, the data may be converted to an event in a music-based format using the musical instrument digital interface (MIDI) protocol. In another embodiment, the data may be converted to an event in the form of a USB HID Keyboard command which may then be used to control a music-based computer game.

The music-based interface may provide an assessment of hand function in real-time and/or at a different time, such as at the end of a song. The assessment may provide 1) motivation to those performing therapy through auditory and visual feedback, 2) a quantitative measure of hand function for research or clinical purposes, and/or 3) the ability to tailor the level of difficulty and type of therapy based on an assessment of hand function both in real-time and some point in time, such as after each song.

The assessment may be presented to the individual performing therapy in the form of auditory and visual cues representing the timing and correctness of the intended movement. Other aspects of hand and finger movement, such as force and velocity, may also be represented in the form of auditory or visual cues. The level of difficulty may be adjusted based on the assessment. Changing the difficulty may include increasing the complexity of notes being played and narrowing the timing window where a note is considered correctly played.

After each song, a quantitative measure of hand function may be provided including the total number of notes hit, total number of notes hit on each digit, timing information on the notes hit correctly and incorrectly, consecutive number of correct notes hit, the magnitude of force exerted by each individual finger and all fingers, and the duration of the song. This immediate quantitative feedback following each song provides motivation to the patient. The data associated with the assessment of the hand motor function may be stored onto a computer, server, or the like and be accessible locally or remotely over a network of one or more interconnected devices, such as the Internet, a private network, LAN, WAN, and/or MAN. The data may enable clinicians and patients to view the assessments from any location that is configured to access the logged data. Any number of devices may be used to access the logged data including a mobile device, a computer, a laptop, or other similar device. The data may be accessed wirelessly or using a fixed wire connection. The music-based interface may be in communication with any type of device including a computer, laptop, or mobile device.

Logging a quantitative measure of hand function may provide clinicians and/or researchers an objective view of motor improvement over time, an objective view of the amount of guided or unguided exercise over time, and/or incentive for the patient to improve in duration of exercise and assessment scores.

The disclosed methods may also be applied to other treatment options including stem-cell therapy, surgical procedures, pharmaceuticals, other therapeutic devices and/or implantable devices.

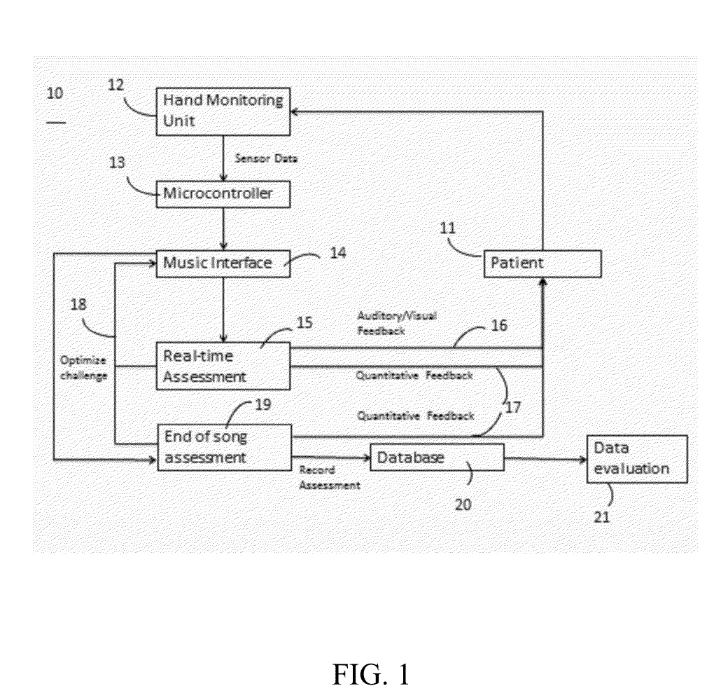

FIG. 1 is a schematic diagram of a music-based rehabilitation system in an embodiment and is shown generally at 10. A patient 11 may be coupled to a hand monitoring unit 12 that may be configured to sense a completed hand movement. A microcontroller 13 receives digital or analog incoming sensor data associated with the hand movement, interprets the data, and sends a signal to a music interface 14. As discussed above, the data may be a MIDI signal and/or a USB HID Keyboard signal. The music interface 14 may support and communicate with any operating system including Windows® and Mac OS®. While performing a song, the patient 11 may receive a real-time assessment 15 of a hand function in the form of auditory and/or visual feedback 16 or quantitative feedback 17. The real-time assessment 15 may be used to motivate the patient 11 and to optimize the challenge or change the level of difficulty of a selected song 18. A song assessment 19 may be presented at the end of each song in the form of quantitative feedback 17 to the patient 11. The feedback 17 may also be provided at any other time as suitable and may be stored in a database 20 or other memory device capable of storing the data. The data may then be evaluated 21 by a clinician/researcher and/or patient or be further processed by other software and hardware.

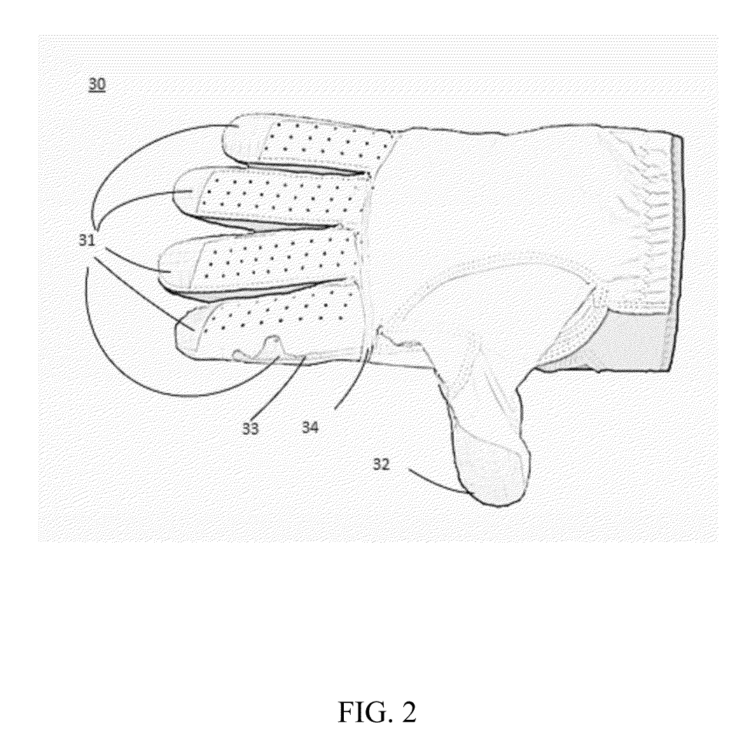

FIG. 2 is a diagram which shows an embodiment of a hand monitoring unit shown generally at 30. Conductive leads 31, 32 may be mounted on a glove. The leads 31, 32 may be coupled to hand monitoring unit 30 in a variety of ways so long as the leads are capable of generating electrical signals associated with a movement. In one embodiment, the glove includes six electrical leads located on all five fingertips and one on the proximal interphalangeal joint on the lateral aspect of the index finger. When the lead on the thumb 32 touches any of the other five leads 31, an electrical connection is closed and registered by a microcontroller as an event.

The conductive leads 31, 32 may be coupled to conductive traces 33—composed of conductive fabric or conductive thread—which may be coupled either directly to pins of a microcontroller or may be soldered onto electrical wire 34 which may then be coupled to a microcontroller. The conductive fabric may be attached to the glove through an adhesive or sewed into the fabric of the glove. The conductive leads may be coupled to the conductive traces either by soldering the two together or sewing the conductive trace into the conductive lead. The conductive fabric may be selected such that it is an aesthetic and flexible conductive material with a large contact surface area. Example materials for the fabric may include nickel and/or copper plated polyester fabric.

In another embodiment, the hand monitoring unit may include a worn system, such as a glove, containing a pressure sensor and a color light sensor positioned on the thumb, and different color material positioned at specific pad locations 31. The color sensor, positioned on the thumb, may recognize which pad location 31 the thumb is coming in contact with based on the specific color of the material. The pressure sensor positioned on the thumb may recognize when contact is made and also the force of the thumb on the pad location 31. In another embodiment, worn system, such as a glove, may include a pressure sensor and/or a magnetometer sensor on the thumb, and different strength magnets positioned at specific locations 31. The magnetometer may register which location 31 the thumb is coming in contact with based on a unique magnetic field strength. The pressure sensor may recognize when contact is made and the force of the thumb on the respective digit.

Another example may include a camera based system which may recognize when functional movements—such as pincer grip, key-pinch grip, and finger-thumb opposition—are completed. This may be used in combination with one or more of the other hand monitoring embodiments described herein

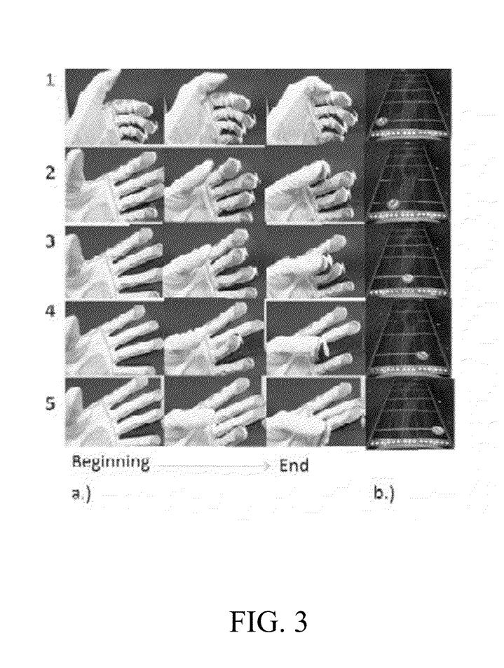

FIG. 3 shows an example of one or more functional grips that may be trained. The five hand movements being trained are shown in three frames each progressing from beginning to end. When an electrical connection is made between the lead on the thumb and one of the other 5 electrical leads, a distinct event is sent to the computer through the USB port. Referring to the right side of FIG. 3, there is shown an example of interfacing with a music-based computer game in which notes denoted by a dot scroll down the screen on five distinct frets. When the notes reach the bottom of the screen, the user must touch the thumb to one of the five respective leads on the glove within a specified time frame. In one embodiment, more notes may be added by combining finger movements or placing more leads on the hand monitoring unit or by using sensing of hand orientation to influence note selection.

The quantitative assessment shows the number of correct notes hit on each fret, total number of correct notes hit, and an average of how close in time the individual was to hitting the note at the correct time. Specifically, the timing term may be calculated as:

$$\overline{X} = 1 \text{/} n {\sum }_{i = 1}^{n} {X}_{i}$$

Where X=deviation in time of a correctly hit note (has to be within 300 ms window to be considered correct), and n=the total number of correctly hit notes in a level.

In the above example, a controller that transmits MIDI signals through USB may be used to enable patients to control software applications that support the MIDI protocol through the glove interface. The controller supports up to 32 simultaneous digital or analog inputs allowing the patient to play any or all notes concurrently. A sample rate of up to 500 Hz may be achieved. The controller may be coupled to a computer through a Universal Serial Bus (USB) port. The USB-MIDI controller may be compatible with all versions of Mac OS X® and Windows® operating succeeding Windows 2000®.

The glove may be designed to providing meaningful exercises that will help the patient perform everyday tasks. Although the human hand is complex in structure with 27 degrees of freedom, a small number of combined joint motions or synergies may account for more than 90% of the variance. Using principle component analysis, everyday tasks can be characterized through nine distinct synergies. The five movements shown in FIG. 3 incorporate six of the nine main synergies, and therefore, provide movement that may be applied to everyday activities.

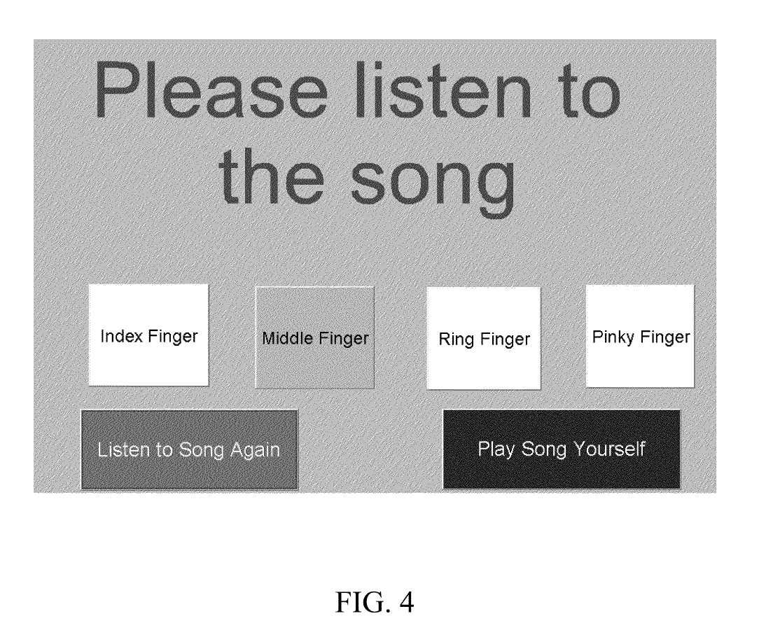

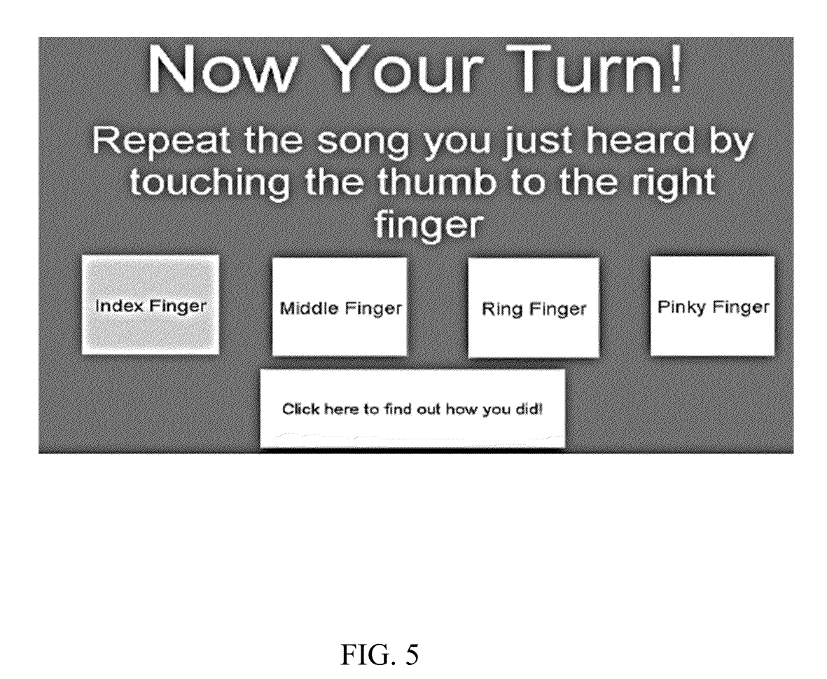

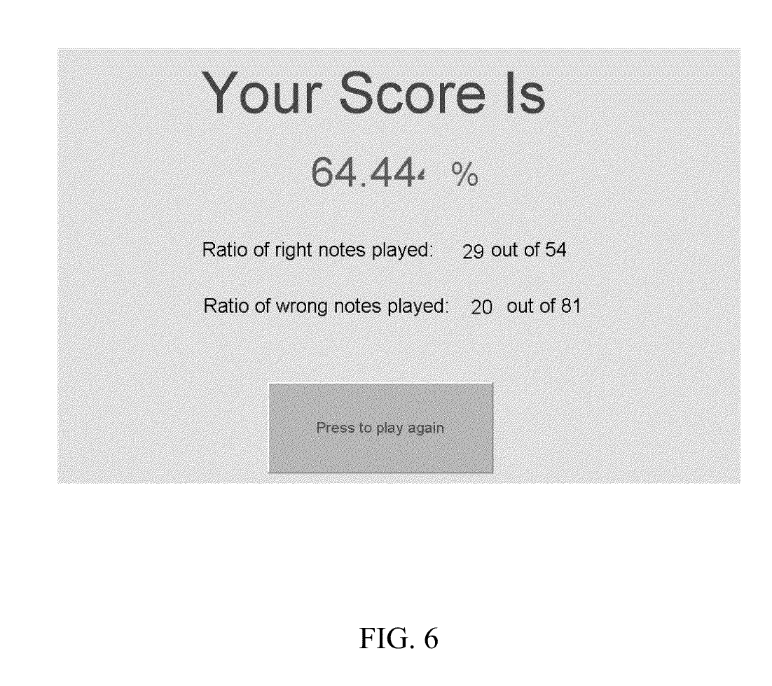

In another example as shown in FIGS. 4-6, the patient may play a computer program which quantitatively measures how well a patient plays a song sample. In the computer game, the patient first listens to a sample of a song which concurrently provides visual feedback on which finger must be pressed with respect to each note. Next, the patient plays the song with visual feedback on which note is being activated. Consequently a score of 0-100% is displayed.

The score may calculated by comparing a patient's performance with a template. The score may be dependent on the note correctness (α), the change in onset of note from the template in milliseconds (Δλ), and the change in duration of note from the template in milliseconds (Δβ). These three factors are evaluated for each note.

The score can be expressed as:

$$Score = 100 - {\sum }_{n = 1}^{K} ( 1 - α ) [ {C}_{1} [ Δ {γ}_{n} ) - {C}_{2} ( Δ {β}_{n} ) ] - \frac{100 α}{κ} \qquad ( 1 )$$

The multiplication factors C₁ and C₂ are adjusted to determine how significant the change in onset and change in duration of the note is with respect to the score.

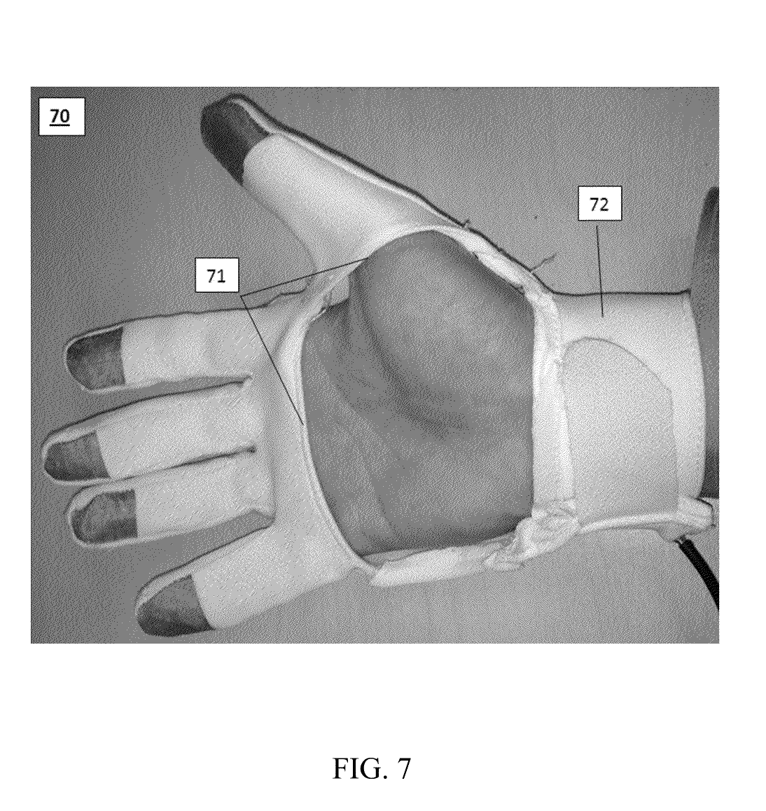

FIG. 7. illustrates a an embodiment of an open palm glove design intended for easy donning and doffing shown generally at 70. The front of the glove 70 may include a large open area in the palmar region 71. This configuration allows one or more digits to be placed into the glove seperately. The glove 70 may also have a wrist component 72 which allows the glove 70 to be fastened onto a hand securely.

The present invention or any part(s) or function(s) thereof, may be implemented using hardware, software, or a combination thereof, and may be implemented in one or more computer systems or other processing systems. A computer system for performing the operations of the present invention and capable of carrying out the functionality described herein can include one or more processors connected to a communications infrastructure (e.g., a communications bus, a cross-over bar, or a network). Various software embodiments are described in terms of such an exemplary computer system. After reading this description, it will become apparent to a person skilled in the relevant art(s) how to implement the invention using other computer systems and/or architectures.

The foregoing description of the preferred embodiments of the present invention has been presented for purposes of illustration and description. It is not intended to be exhaustive or to limit the invention to the precise form or to exemplary embodiments disclosed. Obviously, many modifications and variations will be apparent to practitioners skilled in this art. Similarly, any process steps described might be interchangeable with other steps in order to achieve the same result. The embodiment was chosen and described in order to best explain the principles of the invention and its best mode practical application, thereby to enable others skilled in the art to understand the invention for various embodiments and with various modifications as are suited to the particular use or implementation contemplated. It is intended that the scope of the invention be defined by the claims appended hereto and their equivalents. Reference to an element in the singular is not intended to mean “one and only one” unless explicitly so stated, but rather means “one or more.” Moreover, no element, component, nor method step in the present disclosure is intended to be dedicated to the public regardless of whether the element, component, or method step is explicitly recited in the following claims. No claim element herein is to be construed under the provisions of 35 U.S.C. Sec. 112, sixth paragraph, unless the element is expressly recited using the phrase “means for . . . . ”

It should be understood that the figures illustrated in the attachments, which highlight the functionality and advantages of the present invention, are presented for example purposes only. The architecture of the present invention is sufficiently flexible and configurable, such that it may be utilized (and navigated) in ways other than that shown in the accompanying figures.

Furthermore, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the present invention in any way. It is also to be understood that the steps and processes recited in the claims need not be performed in the order presented.

1. An apparatus for providing rehabilitation, comprising:
a hand monitoring unit configured with one or more electrical signals associated with a completion of at least one hand movement; and
a music-based interface configured to receive the one or more electrical signals from the hand motoring unit representative of the at least one hand movement.

2. The apparatus of claim 1, wherein the at least one hand movement further comprises one of a pincer grip, key-pinch grip, and/or finger-thumb opposition.

3. The apparatus of claim 1, wherein the music-based interface is configured to communicate with one of a mobile device, a laptop, and a computer.

4. The apparatus of claim 3, wherein the music-based interface is configured to provide one of visual and audio feedback when at least one hand movement corresponding to a musical note is completed within a target window of time corresponds to a playing of the musical note by the music-based interface.

5. The apparatus of claim 1, wherein the hand monitoring further comprises a glove configured with a plurality sensors.

6. The apparatus of claim 4, wherein the glove further comprises electrical leads configured on one or more fingertips of the glove.

7. The apparatus of claim 1, wherein the music-based interface is configured as a MIDI interface.

8. The apparatus of claim 1, wherein the music-based interface is configured to one of quantify, record, and store the one or more electrical signals from the hand monitoring unit.

9. The apparatus of claim 1, wherein the music-based interface is configured to provide an assessment of sensory motor ability through one of audio and video feedback.

10. The apparatus of claim 1, wherein the hand monitoring unit is configured to translate data into a USB HID Keyboard command.

11. The apparatus of claim 1, wherein the hand monitoring unit comprises one of conductive fabric and conductive thread.

12. The apparatus of claim 1, further comprising one or more traces coupled to a microcontroller.

13. The apparatus of claim 5, wherein the glove is configured to facilitate one of donning and doffing.

14. The apparatus of claim 8, wherein the fabric is one of nickel and copper plated polyester.

15. A method for providing rehabilitation, comprising:
providing a hand monitoring unit configured with one or more electrical signals associated with a completion of at least one hand movement; and
providing a music-based interface configured to receive the one or more electrical signals from the hand motoring unit representative of the at least one hand movement.

16. The method of claim 15, wherein providing the music-based interface further comprises configuring the music-based interface to communicate with one of a mobile device, a laptop, and a computer.

17. The method of claim 15, wherein providing the music-based interface further comprises configuring the music-based interface to provide one of visual and audio feedback when at least one hand movement corresponding to a musical note is completed within a target window of time corresponds to a playing of the musical note by the music-based interface.

18. The method of claim 15, wherein providing the hand monitoring unit further comprises providing a glove with a plurality sensors.

19. The method of claim 15, wherein providing the music-based interface further comprises configuring the music-based interface as a MIDI interface.

20. The method of claim 15, wherein providing the music-based interface further comprises configuring the music-based interface to provide an assessment of sensory motor ability through one of audio and video feedback.
